(19) United States
(12) Patent Application Publication
(10) Pub. No.: US 2020/0093985 A1
(43) Pub. Date: Mar. 26, 2020

(54) STRAP FOR SECURING A MEDICAL APPLIANCE TO A PATIENT

(71) Applicant: Mark G. Greenberg, Oakland, NJ (US)

(72) Inventor: Mark G. Greenberg, Oakland, NJ (US)

(21) Appl. No.: 16/580,496

(22) Filed: Sep. 24, 2019

Related U.S. Application Data
(60) Provisional application No. 62/737,102, filed on Sep. 26, 2018.

(51) Int. Cl. A61M 5/158; A61M 25/02

(52) U.S. Cl. CPC A61M 5/158; A61M 25/02; A61M 2205/0216; A61M 2005/1586; A61M 2025/0246; A61M 2205/0238; A61M 2025/026

(57) ABSTRACT

A disposable noninvasive strap is disclosed for use in securing a medical appliance such as an I.V. line, gauze pad, or the like, to a patient. The disposable strap is removably secured to the patient, and in a particular embodiment, may be applied to a patient's torso or a limb, such as a wrist, arm or leg, and holds the medical appliance without undesirable movement. An exemplary strap comprises a layer of soft, porous, elastomeric material and particularly, is prepared from a spun bond laminate of polypropylene. In a further exemplary embodiment, the strap may applied to the site of a venipuncture after the withdrawal of the needle, to hold in place a cotton or gauze pad or the like, that is applied to the site to absorb and curtail the flow of any blood or other fluid from the same.

### CROSS-REFERENCE TO RELATED APPLICATION

This application claims priority to U.S. Provisional Patent Application No. 62/737,102, filed on Sep. 26, 2018, the contents of which are incorporated herein by reference in its entirety.

### BACKGROUND OF THE INVENTION

The present invention relates generally to a device for securing one or more medical appliances to a patient, and more particularly, to a disposable noninvasive strap to hold devices such as intravenous (I.V.) lines, medicament dispensers, vital sign sensors, bandage and gauze-like material, and the like, that is used in connection with the administration of medical care to a patient.

### DESCRIPTION OF THE PRIOR ART

In the course of administering medical treatment or care to a patient, whether in a hospital, urgent care facility or other outpatient setting, the patient is likely to require and thereby receive the application to the person of one or more conventional medical appliances, ranging from devices for the examination or monitoring of patient condition to appliances for the administration of medication or other treatment to the person.

Of these, the most common appliances are I.V. tubing associated with the delivery of medical fluids to the person. More particularly, such I.V. tubing, or catheter extends between a container holding a particular medicament or like liquid, which container is usually suspended from a support, and terminates in connection with an infusion device such as an appropriately configured syringe or needle. In practice, the catheter is then secured against the body of the patient at a location and proximity to the insertion site of the syringe or needle. Such securement normally involves the application to the skin of the patient of an extensive expanse of adhesive sheet or strip material to hold the catheter in place during infusion.

A typical procedure follows: From the insertion site, the I.V. tubing is formed into a short loop to the left or right of the insertion site, as applicable, and secured in place with short lengths of adhesive tape. The tubing is then laid out along the length of the body, wrist, leg, etc., and secured with additional lengths of adhesive tape. After the infusion or other procedure is concluded, the syringe or needle, the associated catheter and the adhesive tape holding them against the patient's skin, are removed from the patient. Despite efforts to minimize trauma or discomfort to the patient, the removal process often injures the patient's skin and causes the patient localized pain.

In certain patients having thin skin, for example, removal of the tape tears or otherwise impairs the integrity of the skin, subjecting such patients to subsequent infections, which necessitate additional treatment. Hypoallergenic components prevent allergic rashes and irritation, which are commonly experienced with the application of conventional medical adhesive tape in securing I.V. tubing to skin. Valuable time is saved by avoiding the use of varied strips of adhesive tape to secure tubing to skin. Tape must frequently be replaced which exponentially increases these problems.

As the patient moves, the tubing is likely to catch on bedding or equipment, or the patient may roll over on it. Each time this happens the tube is jerked, and the insertion site, despite the adhesive tape, is jerked at as well.

In addition to the pain, trauma is exerted on the insertion vein, often leading to vascular spasm at the insertion site. The vein collapses at the site, necessitating removal of the I.V., which is then inserted at a new site. This results in additional patient misery.

Numerous devices are disclosed in the prior art that purport to secure a catheter or like appliance to the body of a patient without causing abrasion or injury to the skin. Two such representative devices are disclosed in U.S. Pat. No. 4,569,348 to Hasslinger and U.S. Publication 2002/0177817 A1 to Daniels. In both instances, the devices comprise a strap that is adapted to encircle the portion of the anatomy of the patient adjacent to the site of insertion of the infusion syringe needle, and that utilize a Velcro®-type closure means to hold the strap in position against the body. Also, both devices utilize a composite strap that has a flexible foam as the interior surface of the strap, i.e. the surface contacting the patient's body.

While the foam interior layer may offer greater comfort to the patient, it has the drawback of lacking the adhesive quality that would assure its stability in use. Further, the manufacture of an essentially composite strip or band with the use of the foam interior layer, would add to the cost of an item that is designed and used as a disposable commodity.

There is accordingly a need for the manufacture and use of a medical appliance securing device that is of simple construction and efficient operation, and that conveys the comfort and freedom from injury that is lacking in the devices presently in use. It is therefore toward the satisfaction of this need that the present invention is directed.

### SUMMARY OF THE INVENTION

The present invention is a single-patient disposable noninvasive strap for use in securing a medical appliance such as an I.V. line, or the like, to a patient. The disposable strap is removably secured to the patient, for example to the body, or to a limb, such as an arm or leg, and holds the appliance without allowing undesirable movement in use or injury or trauma on removal. The disposable strap represents an improvement over the use of adhesive tape to secure a medical appliance such as an I.V. line to a patient.

The strap of the present invention has an adjustable configuration by virtue of the fact that the band comprising the strap is prepared from a nonwoven fibrous material known as a spun bond laminate. The present spun bond laminate comprises a plurality of elastomeric fibers that exhibit the elasticity to stretch to a multiple of their length and width at rest, and to return to the original dimensions when tension is released. The device may be constructed using hypoallergenic components, for particular use with patients having damaged or compromised skin, and secures and stabilizes medical appliances to the patient's body without the use of adhesive material. The device thus preserves skin integrity and prevents unwanted movement or dislodging of the appliance.

Accordingly, in its broadest aspect, the strap of the present invention comprises a monolithic band prepared from a non-woven fibrous elastomeric material, the band having a first outer broad surface defining the exterior of the strap in use, and a second inner broad surface that directly contacts the patient in use and that enters into releasable non-slip engagement with the skin of said patient, and fastening means located on said outer broad surface adjacent the terminus of one end of the band, for securing the band to the patient and for securing the medical appliance against the band.

The fastening means comprises a first distal fastener for securing the band to the patient, a second proximal fastener spaced apart from the first fastener and closer to the terminus, for securing the medical appliance against the band. Both the first and the second fasteners are prepared from and comprise Velcro®-type hook materials. The hook materials are disposed on a base or sheet, which base or sheet may be a non-woven polymeric material. Thus, in use, the hook materials of the distal fastener are able to securely grasp the surfaces of the band that are adjacent to that fastener when the band is first applied to the patient, and subsequently, the hook materials of the proximal fastener engage the respective adjacent portion of the band when the medical appliance is secured against the adhesive and the remainder of the band is placed in contact with the said proximal fastener.

Accordingly, when the strap is first wrapped around the patient, hooks of the hook material disposed on the distal fastener make initial contact with and engage the adjacent surface of the band to hold the strap securely against the patient. The filamentous nature of the nonwoven polymeric material of the band enables it to function like the loop material found in Velcro®, and to form a releasable engagement with the hook material when the two are brought together.

An adhesive surface is located on the outer broad surface of the band, intermediate the first and second fasteners. The adhesive surface is provided to grasp and hold the medical appliance to be secured with the use of the strap. The adhesive surface preferably has a protective layer that covers the adhesive before use and which is removed to expose the adhesive. In the instance where the medical appliance to be attached is an I.V. line, and as described and illustrated herein, the line is laid on the exposed adhesive and the adjacent surface of the band is drawn over the I.V. to secure it to the strap.

In particular embodiments, the adhesive surface may be a coating that is applied directly to the exposed area of the outer broad surface of the band that lies intermediate the first and second fasteners. Alternately, the adhesive surface may be disposed on a pad that in turn, is attached to the exposed area of the outer broad surface. The pad may be prepared from a strip of polymeric material, and may comprise a sponge or foam. In a particular embodiment, the pad may be prepared with the adhesive applied to both broad surfaces thereof, so that it is adhesively bound to the first broad surface.

In addition to its use for the securement of medical appliances associated with examination and treatment, the strap of the present invention is particularly advantageous for application to secure a gauze pad or the like, that is conventionally applied to the location of a venipuncture after the conclusion of an infusion procedure and the withdrawal of the needle and catheter. In the majority of cases, the strap is already generally positioned on the patient adjacent to the point of venipuncture to secure the I.V. line during infusion. In some instances, the strap has been removed at the conclusion of infusion and would need to be reapplied. Accordingly, when the needle is withdrawn, a gauze pad or the like is applied to the site of the withdrawn needle to prevent blood loss. At that point, and in the instance where the strap is still in place on the patient, the strap may be easily moved in the direction of the gauze pad and expanded so as to facilitate its placement over such pad and its subsequent elastic contraction and retention of the pad in place over the site of puncture. In the instance where the strap had been removed, it may be positioned over the gauze pad and reapplied.

The porous and elastic nature of the non-woven elastomeric material constituting the monolithic band permits the strap to breathe and allow oxygen to get to the patient's skin, and prevents the strap from moving along the limb or other part of the body around which the strap is applied.

Accordingly, it is a principal object of the present invention to provide a means for securing a medical appliance to a patient that is of simple construction and avoids the injury and trauma of adhesive tape directly applied to the patient body.

It is a further object of the present invention to provide a securing means as aforesaid that is inexpensive to manufacture and use, and that is easily disposable.

It is a still further object of the present invention to provide a securing means comprising a disposable strap for the securement of a medical appliance that is sufficiently elastic to accommodate a variety of sizes of patients and locations for placement on the patient's body.

Other objects and advantages will become apparent from a consideration of the ensuing description, which proceeds with reference to the following illustrative drawings.

### BRIEF DESCRIPTION OF THE DRAWINGS

FIG. 1 is a top view of the disposable strap in accordance with the present invention;

FIG. 2 is a side view of the disposable strap;

FIG. 3 is a bottom view of the disposable strap;

FIG. 4 is a fragmentary perspective view showing the first step in the use of the strap of the present invention, where the strap has been wrapped and secured on the arm of a patient preliminary to the insertion of an I.V. line and needle therein;

FIG. 5 is a fragmentary perspective view showing the preparation of the strap to receive the IV line, by removing the cover from the adhesive surface;

FIG. 6 is a fragmentary perspective view similar to FIG. 5 showing the placement of the I.V. line along the exposed adhesive surface;

FIG. 7 is a fragmentary perspective view similar to FIGS. 5 and 6, showing the completion of the installation of the strap by the securement of the I.V. line against the adhesive surface, by drawing the loose end of the band across the adhesive surface and attaching the end of the band to the first fastener;

FIG. 8 is a fragmentary perspective view similar to FIG. 4, showing the placement of a pad against the site of venipuncture to prevent the flow of fluid from the site; and

FIG. 9 is a fragmentary perspective view similar to FIG. 8, showing the site of venipuncture secured against fluid flow by the placement of the strap of the invention over the pad.

### DETAILED DESCRIPTION OF THE INVENTION

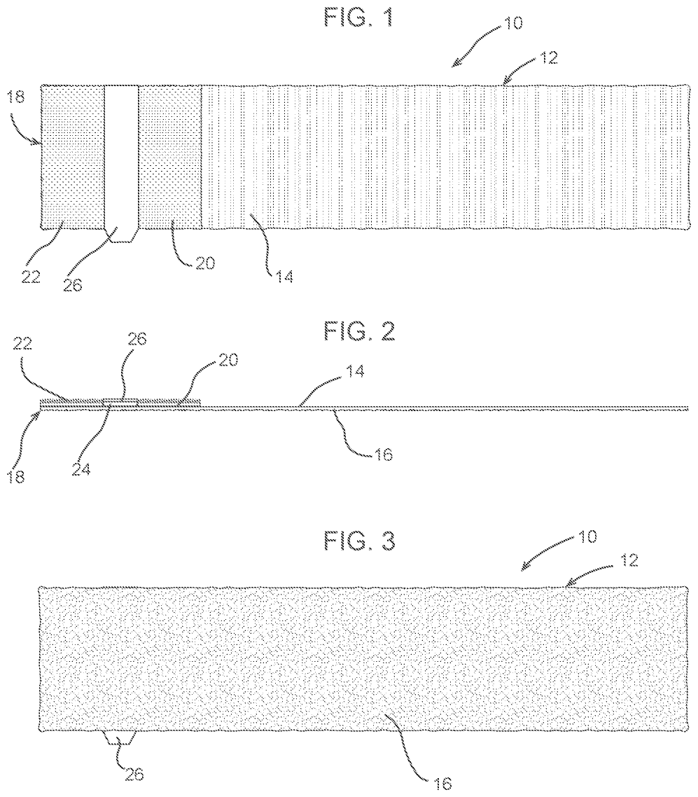

Referring now to the figures wherein like numerals designate like parts, and to FIGS. 1-3, a strap 10 for the securement of a medical appliance such as a catheter, medication dispenser, cotton or gauze pad or the like, to a patient is disclosed. Strap 10 comprises a monolithic band 12 that is prepared in its entirety from a non-woven elastomeric material. More particularly, band 12 is prepared from a material known as a spun bond laminate. The spun bond laminate of the present invention may be made in a continuous process, where fibers are spun and then directly dispersed into a web by deflectors or directed with air streams. A discussion of spun bond laminates can be found, for example, in U.S. Pat. No. 5,952,252 to Shawver, et al. and patents cited therein, all incorporated herein by reference and made a part hereof. This material has the necessary properties for stretch and fastening that are required by the present strap.

The spun bond laminates useful in the present invention can be prepared from a variety of organic polymeric materials, including by way of non-limiting example, polyesters, polycarbonates and polyolefins. In a particular embodiment, the present spun bond laminate may be prepared from polyolefins, and more particularly, from one or more polyalkylenes. In a particular exemplary embodiment, the spun bond laminate may be prepared from an elastomeric polymeric material comprising or consisting of polypropylene.

Referring now to FIGS. 1 and 2, band 12 has a first outer broad surface 14 defining the exterior of the strap in use, and a second inner broad surface 16 that directly contacts the patient in use and that enters into releasable non-slip engagement with the skin of said patient. Fastening means comprising a first distal fastener 20 and a second proximal fastener 22 are located on outer broad surface 14 adjacent terminus 18, one of the ends of band 12. First distal fastener 20 is spaced apart from second proximal fastener 22 and separated therefrom by adhesive surface 24 disposed adjacent and between the said fasteners.

Referring further to FIG. 2, adhesive surface 24 is located and disposed on outer broad surface 14 between distal fastener 20 and proximal fastener 22. Adhesive surface 24 is provided to grasp and hold the medical appliance to be secured with the use of strap 10. Adhesive surface 24 may be a coating that is applied directly to the exposed area of outer broad surface 14. In another embodiment, adhesive surface 24 may be disposed on a pad, not specifically depicted herein, that in turn, is attached to outer broad surface 14. The pad may be prepared from a strip of polymeric material and may comprise, for example, a sponge or foam. In a particular embodiment, the pad may be prepared with adhesive applied to both broad surfaces thereof so that it is bound to the outer broad surface 14 and defines on its opposite broad side, adhesive for the reception and securement of the medical appliance.

Referring again to FIG. 2, a protective layer or strip 26 is disposed over adhesive surface 24 to protect surface 24 from unwanted adhesion or even loss of adhesive capability by atmospheric exposure prior to its intended use. In a particular embodiment, layer or strip 26 is prepared from an inert flexible material and may have a release coating not shown, on the surface that contacts adhesive surface 24. As described hereinbelow, layer or strip 26 will be removed during the process of applying strap 10 to the patient and securing the medical appliance thereto, to enable the medical appliance such as an I.V. line, to be placed against adhesive layer 24 and secured initially in position thereby.

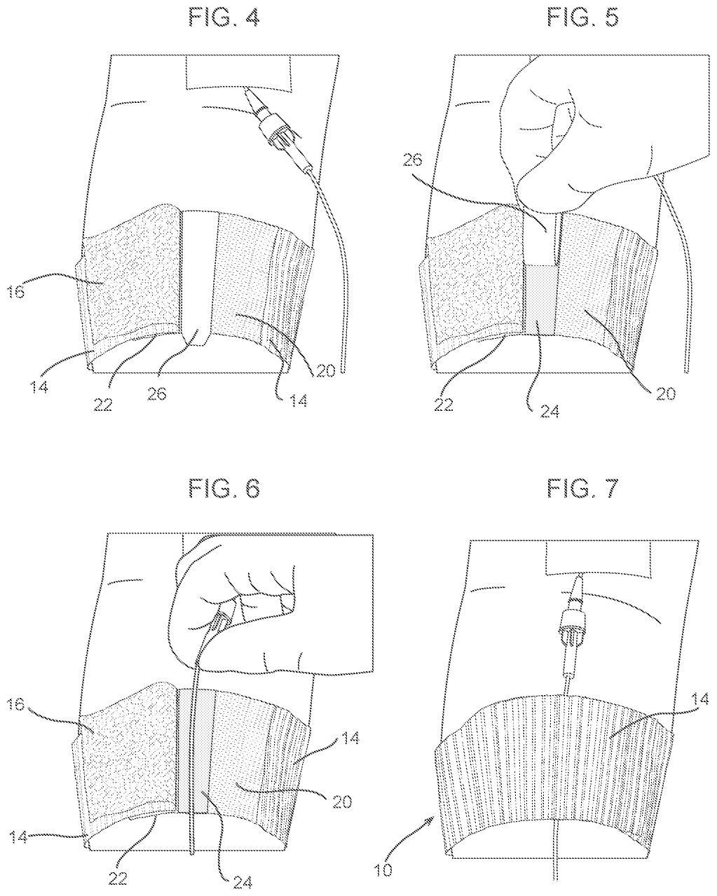

Referring now to FIGS. 4-7, the use of the strap of the present invention in the securement of an I.V. line is illustrated and will be described. Referring first to FIG. 4, the I.V. line and needle have already been inserted into a vein, and the site of venipuncture has been initially secured with a small strip of tape. As illustrated in the figure, strap 10 has been wrapped around the portion of the body adjacent to the site of venipuncture and has been initially secured by the engagement between distal fastener 22 and the adjacent portion of inner broad surface 16.

Referring now to FIG. 5, cover or strip 26 is next peeled away to expose adhesive surface 24. Referring next to FIG. 6, the I.V. line is placed in position along adhesive surface 24 after the complete removal of strip 26. As discussed above, the adhesive coating disposed on adhesive surface 24 grasps and holds the I.V. line in place and enables the completion of the securement with strap 10. Lastly, and as depicted in FIG. 7, the loose portion of strap 10 shown in FIGS. 4-6, that has been labeled in the figures with numeral 14 to identify the visible portion of inner broad surface 16, is drawn firmly across adhesive surface 24 and into engagement with proximal fastener 20, to complete the installation of strap 10 and the securement of the I.V. line in place against the arm of the patient.

As mentioned earlier, the strap of the present invention is particularly versatile in view of the manner in which it secures appliances and the like to the patient's body. In particular, and as described herein, the strap is able to stably secure an appliance against the body without the use of an adhesive, or adhesive tape. As a consequence, the present strap can be moved to another location on the patient body without the trauma associated with tape removal, and likewise, by its construction, retains its capability to be reused and to secure and hold appliances against the body despite previous assembly and disassembly in use. A particular such reuse is the application of strap 10 to the securement of a cotton or gauze pad 28 that is conventionally applied to the site of venipuncture after the withdrawal of the catheter and needle following the completion of an infusion or the like.

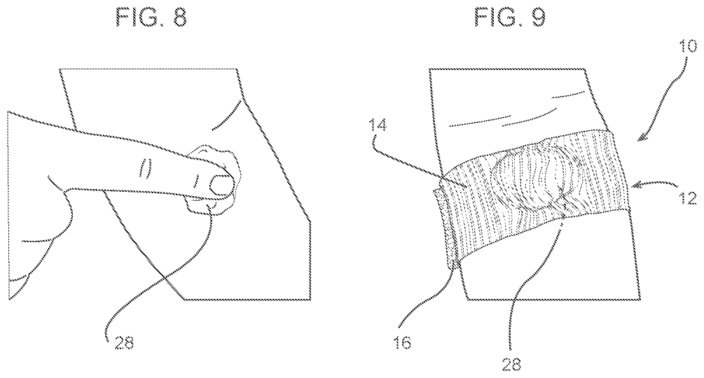

Referring now to FIG. 8, it can be seen that the needle has been withdrawn from the arm and a cotton or gauze pad 28 has been placed against the puncture site and is held in place by a finger. As shown in FIG. 9, strap 10 has been placed over the site where the cotton or gauze pad 28 was positioned and held, and securely retains pad 28 against the puncture site. While not shown in detail herein, strap 10 may be initially opened by the disengagement of the portion of inner broad surface 16 secured to proximal fastener 22, at which point the I.V. line may be removed from adhesive surface 24. Strap 10 thus partially opened, may be moved from its location where it held the I.V. line, as shown in FIG. 7, in the direction of the puncture site, where it may be expanded to place it into position over pad 28, following which it is permitted to contract around pad 28, and the free end of band 12 is drawn firmly across the location of pad 28 to secure the same against the site, as shown in FIG. 8. In an alternate procedure to that just described, strap 10 may be removed from the patient in its entirety, and then reinstalled over pad 28 following the placement of pad 28 against the puncture site, as illustrated in FIG. 8.

From the above description, it should be apparent that the disposable monolithic strap of the present invention is of simple and inexpensive construction and correspondingly simple and reliable operation, and possesses the ability to secure medical appliances such as I.V. lines without the need for extensive use of adhesive tape and corresponding trauma and injury to the patient. The inner broad surface 16 of strap 10 provides a secure yet comfortable engagement with the skin of the patient, so that the appliance to be secured is maintained without unwanted motion and is correspondingly easy to remove. As described, the steps for the installation and removal of strap 10 are simple and painless, and the nature of the strap assures its reliability even in reuse, and thereby further commends its cost effectiveness.

Thus, a disposable strap for securing a medical appliance such as an I.V. line to a patient has been disclosed. It is to be understood that the described embodiment is merely illustrative of some of the many specific embodiments that represent applications of the principles of the present invention. Accordingly, numerous and other arrangements can be readily devised by those skilled in the art without departing from the spirit and scope of the invention.

What is claimed is:

1. A disposable strap for releasably securing a medical appliance to a patient, said disposable strap comprising:
a monolithic band prepared from a non-woven fibrous elastomeric material, said band having a first outer broad surface defining the exterior of the strap in use, and a second inner broad surface that directly contacts the patient in use and that enters into releasable non-slip engagement with the skin of said patient; and
fastening means located on said outer broad surface adjacent the terminus of one end of said band for securing said band to said patient and for securing said medical appliance against said band, said fastening means comprising:
a first distal fastener for securing said band to said patient,
a second proximal fastener spaced apart from said first fastener and closer to said terminus, for securing said medical appliance against said band, and
an adhesive surface located intermediate said first fastener and said second fastener for reception and engagement of said medical appliance thereon.

2. The strap of claim 1, wherein the nonwoven fibrous elastomeric material is a spun bond laminate.

3. The strap of claim 2, wherein the spun bond laminate is prepared from polypropylene.

4. The strap of claim 1, wherein the band is hypoallergenic.

5. The strap of claim 1, wherein the first and second fasteners are Velcro-type hook materials.

6. The strap of claim 1, wherein the second fastener extends to the end of said terminus.

7. The strap of claim 1, wherein the second fastener is located at the end of said terminus.

8. The strap of claim 1, wherein the second fastener extends to and is located at the end of said terminus.

9. The strap of claim 1, wherein the adhesive surface has a protective layer that covers adhesive material and which is removed to expose the adhesive material of the adhesive surface.

10. The strap of claim 1, wherein said adhesive surface is an adhesive coating.

11. The strap of claim 1, wherein said adhesive surface is a pad attached to said first broad surface and having an adhesive layer to receive and engage said medical appliance.

12. The strap of claim 11, wherein said pad has an adhesive layer on both broad surfaces thereof.

13. The strap of claim 9, wherein said protective layer is a removable protective strip overlying said adhesive surface.

14. The strap of claim 13, wherein said protective strip has a release coating on the surface that contacts the adhesive surface.
